(19) United States
(12) Patent Application Publication
(10) Pub. No.: US 2017/0065324 A1
(43) Pub. Date: Mar. 9, 2017

(54) WIRELESS ABLATION CATHETER ASSEMBLY

(71) Applicant: Rowan University, Glassboro, NJ (US)

(72) Inventors: Anilchandra Attaluri, Glassboro, NJ (US); Harel Rosen, Glassboro, NJ (US); Ayre Rosen, Glassboro, NJ (US)

(73) Assignee: Rowan University, Glassboro, NJ (US)

(21) Appl. No.: 15/259,816

(22) Filed: Sep. 8, 2016

Related U.S. Application Data
(60) Provisional application No. 62/215,949, filed on Sep. 9, 2015.

(51) Int. Cl. A61B 18/08; A61B 18/02

(52) U.S. Cl. CPC A61B 18/082; A61B 18/02; A61B 2018/00791

(57) ABSTRACT

A wireless ablation catheter assembly including an ablation catheter having a catheter body extending between a proximal end and a distal end with a magnetically-susceptible area defined along the catheter body between the proximal and distal ends. A magnetic field generator is configured to provide a magnetic field to the magnetically-susceptible area such that the magnetically-susceptible area dissipates heat. A method of ablation is also provided.

### CROSS REFERENCE TO RELATED APPLICATION

This application claims the benefit of U.S. Provisional Patent Application No. 62/215,949, titled “Wireless Ablation Catheter Assembly”, filed Sep. 9, 2015, which is incorporated herein by reference in its entirety.

### FIELD OF THE INVENTION

This invention relates to catheters. More particularly, the invention relates to a wireless ablation catheter assembly and methods of ablation utilizing a wireless ablation catheter.

### BACKGROUND OF THE INVENTION

Ablation is a medical technique used to destroy and/or remove diseased or unnecessary tissue from the body through the application of energy to the tissue. It is used to treat many different medical problems, with exemplary applications being cardiac ablation, endometrial ablation, surface ablation, and liver tumor removal. Ablation procedures are generally minimally-invasive, thereby providing numerous advantages over conventional surgery such as shorter patient recovery times, reduced scar length, lessened risk of infection, lower blood loss, and/or shorter hospital stays.

Ablation catheters are generally known in the surgical art. The most common ablation catheters include radiofrequency ablation catheters and microwave catheters. Presently-used radiofrequency ablation catheters and microwave catheters require the use of coaxial cables or wires to deliver heat energy to the target region, which generally limits the minimum diameter of the catheters. Typical coaxial cables used in connection with radiofrequency ablation catheters and microwave catheters have a diameter of between 0.6 mm to 2.5 mm. The catheters themselves require a working channel therein to house the coaxial cable(s), which, depending on the ablation target location/application, can occupy up to ⅕^{th} to ⅓^{rd} of the total catheter diameter. Thus, the diameter of radiofrequency ablation catheters and microwave catheters is at least partially dictated by the diameter of the coaxial cable(s) passing therethrough, consequently limiting the overall minimum size and flexibility of the catheter.

It is generally known that smaller catheter diameters are preferred, particularly for percutaneous applications, as larger openings or incisions have been associated with increased risk of complications such as bleeding and pneumothorax. Furthermore, smaller and more flexible catheters are advantageous in instances where a vascular route is employed to reach the target ablation site.

Additionally, different ablation procedures are best suited to specific tissue types and ablation sites. For example, high temperature ablation may be more effective on one type of tissue, while low temperature ablation may be more effective on another. However, to achieve both high temperature and low temperature ablation, it is generally known that two separate catheter assemblies must be employed, along with the associated insertion and/or removal procedure(s).

Accordingly, this document describes methods and devices that are intended to address the issues discussed above and/or other issues.

### SUMMARY OF THE INVENTION

In at least one embodiment, the present invention provides a wireless ablation catheter including a tip or balloon or capsule comprising a conductive material, or a plurality of materials, that are magnetically-susceptible. When exposed to an external alternating or time varying magnetic field, the magnetically-susceptible area generates heat.

A wireless ablation catheter assembly according to at least one aspect of the present invention comprises an ablation catheter including a catheter body extending between a proximal end and a distal end, wherein a magnetically-susceptible area is defined on the catheter body at or between the proximal and distal ends. The assembly further comprises a magnetic field generator configured to provide a magnetic field to the magnetically-susceptible area such that the magnetically-susceptible area dissipates heat.

In another embodiment of the present invention, a wireless ablation catheter assembly is disclosed which comprises an ablation catheter including a catheter body extending between a proximal end and a distal end, wherein a multi-layer balloon disposed on the catheter body forms a magnetically-susceptible area on the catheter body at or between the proximal and distal ends of the catheter body. The multi-layer balloon comprises at least a first portion configured to enable high-temperature ablation and a second portion configured to enable low-temperature ablation. The assembly further comprises a magnetic field generator configured to provide a magnetic field to the magnetically-susceptible area such that the magnetically-susceptible area dissipates heat.

In another embodiment of the present invention, a method of ablation is described. The method comprises positioning a catheter in a patient such that a magnetically-susceptible area along a body of the catheter is positioned adjacent to a target area within the patient, and selectively applying a magnetic field to the magnetically-susceptible area such that the magnetically-susceptible area dissipates heat when the magnetic field is applied.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is an elevation view of a wireless ablation catheter assembly in accordance with an exemplary embodiment of the invention.

FIG. 2 is an elevation view of a wireless ablation catheter assembly in accordance with another exemplary embodiment of the invention.

FIG. 3 is an elevation view of a wireless ablation catheter assembly in accordance with another exemplary embodiment of the invention.

FIG. 4 illustrates a portion of an exemplary ablation procedure utilizing the wireless ablation catheter assembly of FIG. 1.

FIG. 5 illustrates another portion of the exemplary ablation procedure utilizing the wireless ablation catheter assembly of FIG. 1.

FIG. 6 illustrates another portion of the exemplary ablation procedure utilizing the wireless ablation catheter assembly of FIG. 1.

FIG. 7 illustrates another portion of the exemplary ablation procedure utilizing the wireless ablation catheter assembly of FIG. 1.

FIG. 8 illustrates another portion of the exemplary ablation procedure utilizing the wireless ablation catheter assembly of FIG. 1.

FIG. 9 is an elevation view of a combined wireless high-temperature ablation and cryoablation catheter in accordance with another exemplary embodiment of the invention.

### DETAILED DESCRIPTION OF THE INVENTION

In the drawings, like numerals indicate like elements throughout. Certain terminology is used herein for convenience only and is not to be taken as a limitation on the present invention. The terms “distal” and “proximal” refer, respectively, to directions closer to and away from a patient or treatment site. The following describes preferred embodiments of the present invention. However, it should be understood, based on this disclosure, that the invention is not limited by the preferred embodiments described herein.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.”

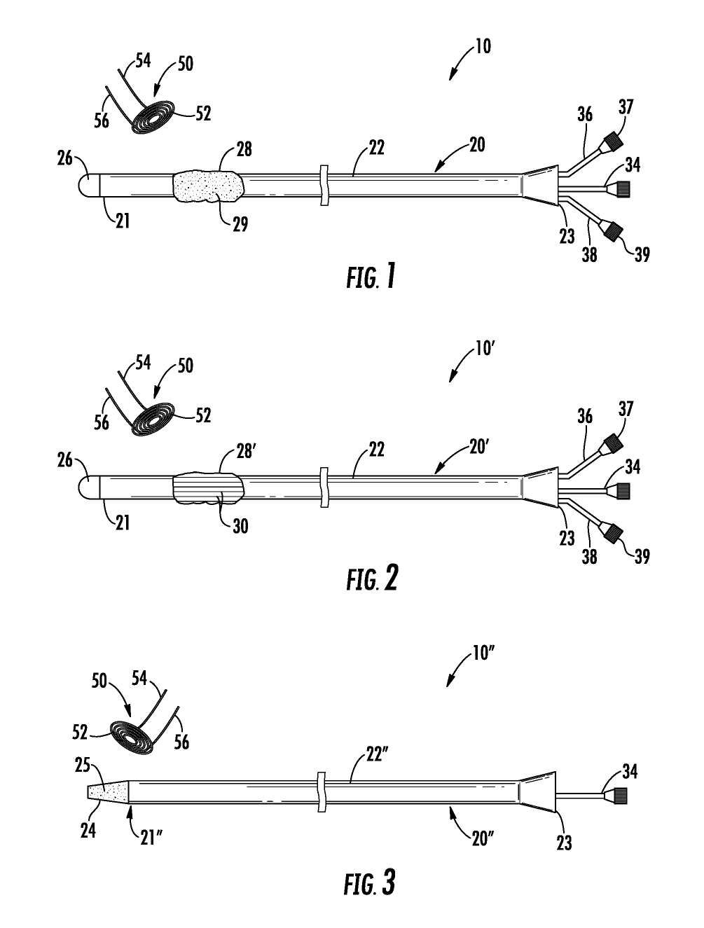

Referring to FIG. 1, an exemplary embodiment of a wireless ablation catheter assembly 10 in accordance with an embodiment of the invention will be described. The wireless ablation catheter assembly 10 generally includes an ablation catheter 20 and a magnetic field generator 50.

The ablation catheter 20 generally includes a tubular catheter body 22 with an internal lumen extending between a distal end 21 and a proximal end 23. The tubular catheter body 22 may be formed of any suitable material, such as a flexible plastic or polymer. In the presently-illustrated embodiment, a positioning balloon 26 extends from the distal end 21 of the catheter body 22 and an ablation balloon 28 is positioned along the catheter body 22 toward the distal end 21. In the present embodiment, the ablation balloon 28 is manufactured from a flexible polymer or similar material, wherein the material contains a plurality of magnetic particles 29 embedded therein such that the balloon 28 defines a magnetically-susceptible area. The balloon 28 may otherwise be manufactured to define the magnetically-susceptible area; for example, the balloon 28 may be coated with a magnetic coating. As another alternative, as illustrated in the embodiment of the wireless ablation catheter assembly 10′ of FIG. 2, the balloon 28′ of the catheter 20′ may have a series of magnetic strips 30 applied to the outer surface thereof. The invention is not limited to the disclosed means of forming the magnetically-susceptible area, and it is to be understood that other means may be utilized.

Additionally, as illustrated in the embodiment of the wireless ablation catheter assembly 10″ of FIG. 3, the catheter body 22″ of the catheter 20″ may be formed without a balloon, and the magnetically-susceptible area may be formed elsewhere on the catheter body 22″. In the illustrated embodiment, the distal end 21″ of the catheter body 22″ has a tapered tip portion 24 which has a magnetic coating 25 applied thereto, with the tip portion 24 designed to contact the tissue at the target site. The tapered tip portion 24 enables the catheter 20″ to puncture or pass through the tissue. However, in some target sites and applications, tip portion 24 is not required to be tapered, as tissue puncture may not be necessary. Thus, tip portion 24 may have non-tapered shapes. Furthermore, in some embodiments, the balloon-less magnetically-susceptible area may be defined elsewhere along the catheter body 22″.

Referring again to FIG. 1, a series of conduits 34, 36 and 38 extend into the catheter body 22 at proximal end 23. In the illustrated embodiment, the conduit 34 is a temperature probe which monitors the temperature adjacent the magnetically-susceptible area. Conduit 36 is associated with the positioning balloon 26. That is, a fluid supply (not shown) is connected to a port 37 of conduit 36 and may be utilized to inflate the positioning balloon 26, as desired. The type of fluid used to inflate positioning balloon 26 is dependent upon the target region and may be, for example, saline or air.

Similarly, conduit 38 is associated with the ablation balloon 28. A fluid supply (not shown) is connected to a port 39 of conduit 38 and may be utilized to inflate the ablation balloon 28, as desired. Again, the supplied fluid is dependent upon the target region and may be, for example, saline or air. It is noted that the catheter 20″ of FIG. 3 does not include the inflation conduits 36, 38 since it does not include either of the balloons. In all other respects, the catheters 20′, 20″ of FIGS. 2 and 3 are generally similar to the catheter 20 of FIG. 1.

In each embodiment described above, the catheter 20 may include additional conduits, including those configured for delivering one or more therapeutics or diagnostics, e.g., cryofluids, chemotherapeutics, ethanol, radiation therapy, imaging agents, immune modulating agents, simultaneously or sequentially with an ablation procedure. In fact, the catheter 20 itself may be coated with drugs or other agents to aid in chemotherapy, immunotherapy, etc., when the catheter 20 is heated. Additionally, the catheter body 22 may be configured or may include one or more additional conduits to facilitate passage of water or other fluid for cooling of the assembly 10.

In each embodiment described above, the magnetic field generator 50 is embodied as an induction coil 52 having leads 54, 56. As another example, the magnetic field generator may be alternating current magnetic field coil. The invention is not limited to such generators, and any magnetic field generator which generates a sufficient magnetic field to induce a desired energy dissipation by the magnetically-susceptible area may be utilized. The magnetic field generator 50 is preferably external to the patient while supplying the desired magnetic field.

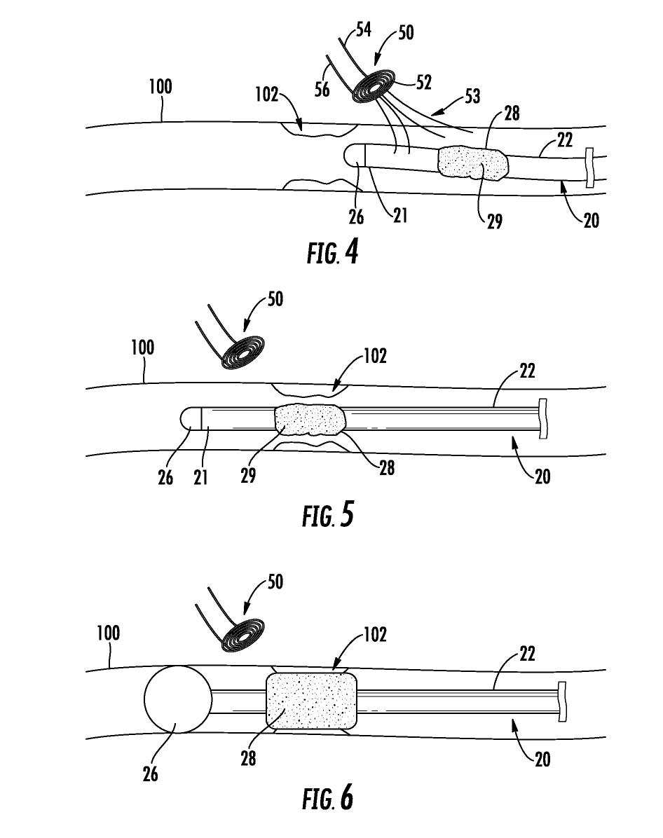

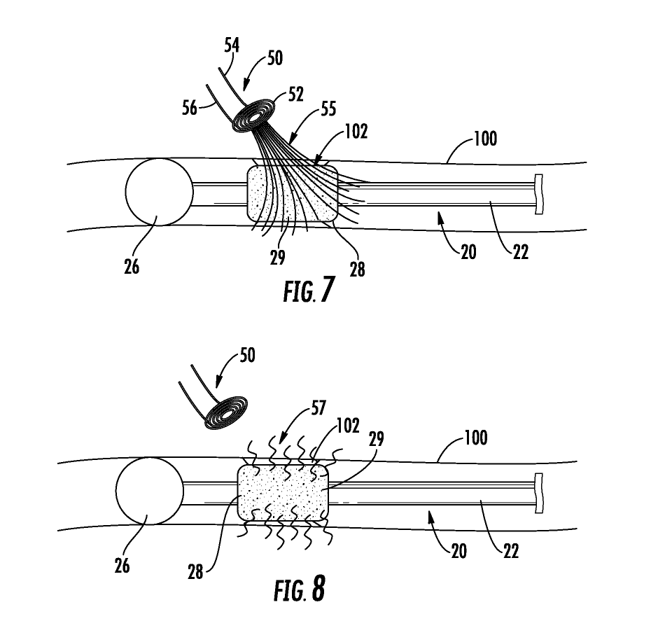

Referring now to FIGS. 4-8, an exemplary ablation procedure utilizing the assembly 10 of FIG. 1 will be described. In FIG. 4, the ablation catheter proximal end 21 is moved within a vessel 100 or the like toward an intended ablation target 102 which comprises, for example, a tumorous growth. As opposed to a blood vessel 100, it is to be understood that the assembly 10 may also be positioned elsewhere within a patient's body. In fact, catheter assembly 10 may be positioned within any body cavity/pathway, either naturally or artificially created. Such body cavities/pathways include, but are not limited to, the nose, nasopharyngeal, trachea, urethra, rectum, blood vessel, etc. As illustrated, since the balloon 28 includes magnetic particles 29, the magnetic field generator 50 may be activated to produce a lower level, static gradient magnetic field 53, which thereby acts on the magnetically-susceptible area and helps to guide the catheter 20 to the ablation target 102. The lower level magnetic field 53 is selected such that it provides attraction, but does not induce significant energy dissipation from the magnetically-susceptible area. The catheter 20 is advanced until the magnetically-susceptible area (in the present illustration, the balloon 28) is aligned with the target area 102, as illustrated in FIG. 5.

Turning to FIG. 6, the positioning balloon 26 is inflated via the conduit 36 (not shown in FIG. 6) such that the positioning balloon 26 engages the wall of the vessel 100 and maintains the position of the catheter 20. The ablation balloon 28 is then inflated with a fluid via the conduit 38 (not shown in FIG. 6) such that the balloon 38 engages the target area 102, whereby the magnetically-susceptible area is in contact with or suitably close to the target area 102.

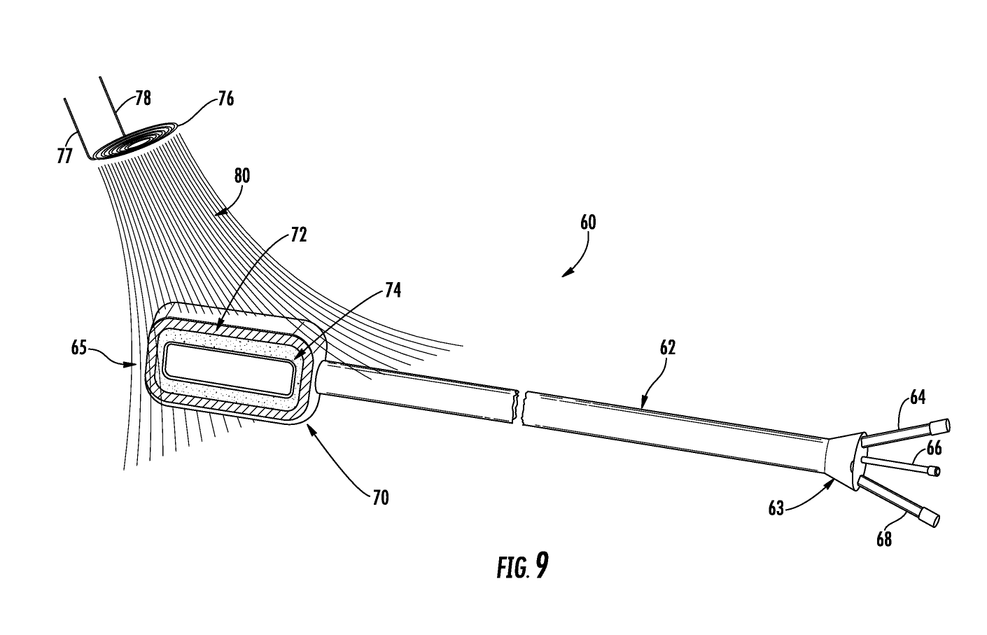

Referring to FIGS. 7 and 8, with the magnetically-susceptible area positioned relative to the target area 102, the magnetic field generator 50 is activated to produce a higher level, alternating magnetic field 55. The higher level magnetic field 55 is such that it induces a desired energy dissipation 57 by the magnetically-susceptible area. Energy dissipation 57 is typically via thermal conduction or convection, dependent upon the application. The magnitude and duration of the energy dissipation 57 may be controlled by the intensity and duration of the application of magnetic field 55. In an exemplary embodiment, the magnitude of the magnetic field 55 may be controlled to be about 0.5 kA/m to 30 kA/m, dependent upon the volume of the heat-generating, magnetic material present on balloon 28. However, the magnitude of the magnetic field is not limited to this range, and the magnitude could be as low as 10 A/m, again dependent upon the volume of the magnetic material on or in balloon 28.

Furthermore, the duration of energy dissipation 57 may also vary dependent upon the particular application. For example, the magnetic field 55 may be applied for only 1 minute or less, or it may be applied for up to 120 minutes. The temperature/thermal dose at the target region may also be controlled, preferably to a temperature between 40°−90° C. It is noted that the thermal dose, like the magnitude of the magnetic field, is also dependent upon the application. As noted above, the conduit 34 comprises temperature probe which monitors the temperature adjacent the magnetically-susceptible area to ensure that the thermal dose does not exceed the desired level for the application.

After the ablation procedure is complete, the catheter 20 is preferably cooled, for example, via liquid flow through the catheter body 22 by way of a separate liquid conduit (not shown). After cooling of catheter 20, the balloons 26, 28 may be contracted, and the catheter 20 removed from the vessel 100.

Referring now to FIG. 9, a dual ablation catheter assembly 60 in accordance with another aspect of the invention is disclosed. Assembly 60 comprises a multi-lumen catheter body 62 extending between a proximal end 63 and a distal end 65. The catheter body 62 may be formed of any suitable material, such as a flexible plastic or polymer.

In the presently-illustrated embodiment, a multi-layer balloon 70 extends from the distal end 65 of the catheter body 62. The multi-layer balloon 70 comprises a first portion 72 configured to contain a conductive material or materials, such as a magnetic fluid, and a second portion 74 configured to contain a cryofluid. The multi-layer balloon 70 may be manufactured from a flexible polymer or similar suitable material. As will be described further hereinbelow, dual ablation catheter assembly 60 enables a single catheter to be used to selectively deliver both extreme high temperatures and extreme low temperatures to an ablation target site. Furthermore, assembly 60 can further be utilized to deliver other therapeutics or diagnostics, such as chemotherapeutics, ethanol, radiation therapy, imaging agents, immune modulating agents, etc., either simultaneously or sequentially.

Similar to assembly 10 described above with respect to FIGS. 1-8, assembly 60 comprises a series of conduits 64, 66, 68 extending into the catheter body 62 at proximal end 63. In the illustrated embodiment, the conduit 66 is a temperature probe which monitors the temperature adjacent the magnetically-susceptible area. Conduit 64 is associated with the first portion 72 of the multi-layer balloon 70. That is, a fluid supply (not shown) is connected to a conduit 64 so as to deliver a conductive material, such as a magnetic fluid, to the first portion 72 of multi-layer balloon 70, thereby inflating balloon 70. Similarly, a cryofluid supply (not shown) may be connected to conduit 68 so as to deliver a cryofluid to the second portion 74 of the multi-layer balloon 70.

When a high-temperature ablation procedure is desired, assembly 60 operates similarly to assembly 10 described above. That is, a magnetic field generator 76 (e.g., an induction coil) having leads 77, 78 is configured to selectively produce a magnetic field 80. As another example, the magnetic field generator 76 may be alternating current magnetic field coil. The magnetic field generator 76 is preferably external to the patient while supplying the desired magnetic field. When located at or near the target ablation site, the magnetic field 80 induces a desired energy dissipation at a magnetically-susceptible area at the conductive material within first portion 72 of balloon 70. Energy dissipation is typically via thermal conduction or convection, dependent upon the application, and the temperature is preferably controlled to a temperature between 40°-90° C. Thus, assembly 60 provides wireless high temperature ablation similar to that described above with respect to assembly 10, as assembly 60 also requires no coaxial cables or similar electrical wires. Such a configuration enables catheter body 62 to have a reduced diameter as compared to conventional radiofrequency ablation catheters and microwave catheters.

In addition to providing high temperature ablation, assembly 60 is also configured to be magnetically steered to the ablation site. As described above with respect to assembly 10, magnetic field generator 76 may generate a lower level magnetic field such that no ablation occurs, but that the multi-layer balloon 70 is capable of being wirelessly steered or led to the ablation site.

In addition to high temperature ablation, assembly 60 is also configured to provide extreme low temperature ablation (i.e., “cryoablation”) in selected instances when the magnetic field 80 is not being produced by the magnetic field generator 76. That is, cryoablation does not occur simultaneously with high temperature ablation, but can occur sequentially, if desired. As set forth above, multi-layer balloon 70 comprises a second portion 74 into which a cryofluid or similar substance may be pumped via conduit 68. The cryofluid generates extremely low temperatures at the ablation site such that a cryoablation procedure is possible. As no coaxial cables or electrical wires are necessary within the catheter body 62 to provide high temperature ablation, there is sufficient space within catheter body 62 for the conduit 68 to pass therethrough without substantially affecting the overall diameter of the catheter body 62.

With the configuration of dual ablation catheter assembly 60 described above and shown in FIG. 9, a single catheter is capable of providing both extreme high and extreme low temperature ablation, where separate catheters (and associated insertion/removal procedures) were previously required. Such ablation procedures may be performed individually (i.e., only high temperature ablation or cryoablation during a given treatment) or sequentially (i.e., high temperature ablation followed by cryoablation, or vice versa).

Furthermore, the catheter assembly 60 may also be configured to deliver one or more therapeutics or diagnostics, either simultaneously or sequentially to the ablation procedure. For example, chemotherapeutics, ethanol, radiation therapy, imaging agents, and/or immune modulating agents may be delivered to the patient at the ablation target site.

The wireless ablation catheter assemblies of the present invention do not require coaxial cables or electrical wires as in the prior art devices. Accordingly, removal of the need for coaxial cables or electrical wires allows for a corresponding reduction in catheter size, simplifies the catheter design, provides for better control of movement and placement, and provides the ability to magnetically steer the catheter via a magnetically-susceptible area on the catheter. Furthermore, as described with respect to FIG. 9, an alternative aspect of the present invention also provides for a dual ablation catheter capable of performing both high temperature and low temperature ablation.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed is:

1. A wireless ablation catheter assembly comprising:
an ablation catheter including a catheter body extending between a proximal end and a distal end, wherein a magnetically-susceptible area is defined on the catheter body at or between the proximal and distal ends; and
a magnetic field generator configured to provide a magnetic field to the magnetically-susceptible area such that the magnetically-susceptible area dissipates heat.

2. The wireless ablation catheter assembly of claim 1, wherein the magnetically-susceptible area is defined by an ablation balloon.

3. The wireless ablation catheter assembly of claim 2, further comprising a conduit extending through the catheter body and in fluid communication with the ablation balloon.

4. The wireless ablation catheter assembly of claim 2, wherein the ablation balloon comprises a magnetic coating thereon.

5. The wireless ablation catheter assembly of claim 2, wherein the ablation balloon comprises a plurality of magnetic strips disposed thereon.

6. The wireless ablation catheter assembly of claim 2, wherein the ablation balloon comprises a magnetic fluid disposed therein.

7. The wireless ablation catheter assembly of claim 1, further comprising a positioning balloon disposed on the distal end thereof.

8. The wireless ablation catheter assembly of claim 7, further comprising a conduit extending through the catheter body and in fluid communication with the positioning balloon.

9. The wireless ablation catheter assembly of claim 1, wherein the magnetically-susceptible area is defined on a distal tip portion of the catheter assembly, further wherein the distal tip portion comprises magnetic elements intended to heat tissue in direct contact with the distal tip portion when the catheter assembly is subjected to the magnetic field.

10. The wireless ablation catheter assembly of claim 1, further comprising a temperature probe extending through the catheter body.

11. The wireless ablation catheter assembly of claim 1, wherein the magnetically-susceptible area is defined by a multi-layer balloon having at least a first portion and a second portion.

12. The wireless ablation catheter assembly of claim 11, wherein the first portion of the multi-layer balloon is in fluid communication with a magnetic fluid source.

13. The wireless ablation catheter assembly of claim 11, wherein the second portion of the multi-layer balloon is in fluid communication with a cryofluid source.

14. The wireless ablation catheter assembly of claim 1, wherein the catheter assembly is further configured to deliver one or more therapeutics or diagnostics.

15. The wireless ablation catheter assembly of claim 14, wherein the one or more therapeutics or diagnostics comprises at least one of chemotherapeutics, ethanol, radiation therapy, imaging agents, and immune modulating agents.

16. The wireless ablation catheter assembly of claim 1, wherein the magnetic field generator is an induction coil.

17. A wireless ablation catheter assembly comprising:
an ablation catheter including a catheter body extending between a proximal end and a distal end, wherein a multi-layer balloon disposed on the catheter body forms a magnetically-susceptible area on the catheter body at or between the proximal and distal ends of the catheter body, further wherein the multi-layer balloon comprises at least a first portion configured to enable high-temperature ablation and a second portion configured to enable low-temperature ablation; and
a magnetic field generator configured to provide a magnetic field to the magnetically-susceptible area such that the magnetically-susceptible area dissipates heat.

18. The wireless ablation catheter assembly of claim 17, wherein the first portion is in fluid communication with a magnetic fluid source and the second portion is in fluid communication with a cryofluid.

19. The wireless ablation catheter assembly of claim 17, wherein the catheter assembly is further configured to deliver one or more therapeutics or diagnostics.

20. A method of ablation comprising:
positioning a catheter in a patient such that a magnetically-susceptible area along a body of the catheter is positioned adjacent to a target area within the patient; and
selectively applying a magnetic field to the magnetically-susceptible area such that the magnetically-susceptible area dissipates heat when the magnetic field is applied.

21. The method of ablation of claim 20, further comprising delivering a fluid to the magnetically-susceptible area along the body of the catheter to position the magnetically-susceptible area adjacent the target area within the patient.

22. The method of ablation of claim 20, further comprising delivering a cryofluid to the magnetically-susceptible area along the body of the catheter positioned adjacent to the target area within the patient when the magnetic field is not applied.

23. The method of ablation of claim 20, wherein positioning the catheter in the patient comprises positioning the catheter within one of a blood vessel and a body cavity.
